(19) United States
(12) Patent Application Publication
(10) Pub. No.: US 2024/0289710 A1
(43) Pub. Date: Aug. 29, 2024

(54) SYSTEM AND METHOD TO FACILITATE IN-PERSON CARES OF ELDER PARENTS BY EMPLOYEES

(71) Applicant: Mirza Muhammad Ajmal Beg, Calgary (CA)

(72) Inventor: Mirza Muhammad Ajmal Beg, Calgary (CA)

(21) Appl. No.: 18/583,860

(22) Filed: Feb. 22, 2024

Related U.S. Application Data
(60) Provisional application No. 63/486,993, filed on Feb. 26, 2023.

(51) Int. Cl. G06Q 10/0631

(52) U.S. Cl. CPC G06Q 10/063116; G06Q 10/063112

(57) ABSTRACT

A system to facilitate in-person care of elder parents comprising comprising: a module to determine opportunities to work remotely and the skills required, a module to determine opportunities to change in-person roles near the elder parents and the skills required, a module to generate skills upgrade plan, a module to manage approval workflow and a module to determine compensation.

### CROSS REFERENCE TO RELATED APPLICATIONS

This application claims priority of U.S. Provisional Application No. 63/486,993 (SYSTEM AND METHOD TO FACILITATE IN-PERSON CARE OF ELDER PARENTS BY EMPLOYEES) filed Feb. 26, 2023 which is further based on U.S. Provisional Application No. 63/314,425 (SYSTEM AND METHOD TO FACILITATE IN-PERSON CARE OF ELDER PARENTS BY EMPLOYEES) filed Feb. 27, 2022. U.S. Provisional Application No. 63/486,993 is incorporated herein by reference.

### BACKGROUND OF THE INVENTION

### Field of the Invention

The present invention is directed to a system, computer-implemented method and storage medium with computer instructions to facilitate one or more in-person cares of one or more elder parents by one or more employees by making it possible for the one or more employees to live near or with the one or more elder parents.

### Description of the Related Art

No system exists which effectively make it possible for one or more employees working away from their one or more elder parents to live near or with their one or more elder parents, to provide one or more in-person cares.

### SUMMARY OF THE INVENTION

In one exemplary embodiment of the present invention, a computer-implemented method and a system are provided that determines one or more opportunities to work remotely for the one or more employees within one or more associated Program Participants and/or other one or more associated Program Participants in need of skills. In exemplary embodiment of the present invention, a computer-implemented method and a system are provided that determines one or more opportunities to work in-person for the one or more employees within one or more associated Program Participants and/or other one or more associated Program Participants in need of skills. In other exemplary embodiment of the present invention, one or more workflows are provided to approve transfer/exchange within the one or more program participants. In other exemplary embodiment of the present invention, a computer-implemented method and system are provided to determine compensation after transfer/exchange. In other exemplary embodiment, a computer-implemented method and system are provided to determine one or more program eligibility criteria. In other exemplary embodiment, a computer-implemented method and system are provided to define and select one or more assignment strategies.

### BRIEF DESCRIPTION OF THE DRAWINGS

FIG. 1 shows a block diagram of an example computer used to provide computing functionalities to implement the present invention. FIG. 2 illustrates an exemplary data model used by the system and the computer-implemented method. FIG. 3 is a diagram conceptually illustrating an exemplary system and the computer-implemented according to the present invention. FIG. 4 is a flow chart illustrating an exemplary operation of enabling one or more transfers and/or one or more exchanges according to the present invention. FIG. 5 is a flow chart illustrating an exemplary one or more transfers and/or one or more exchange strategies according to the present invention.

### DETAILED DESCRIPTION OF THE INVENTION

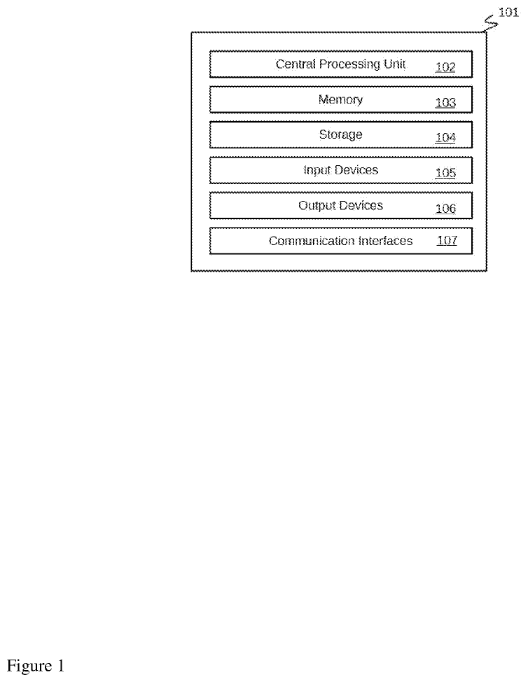

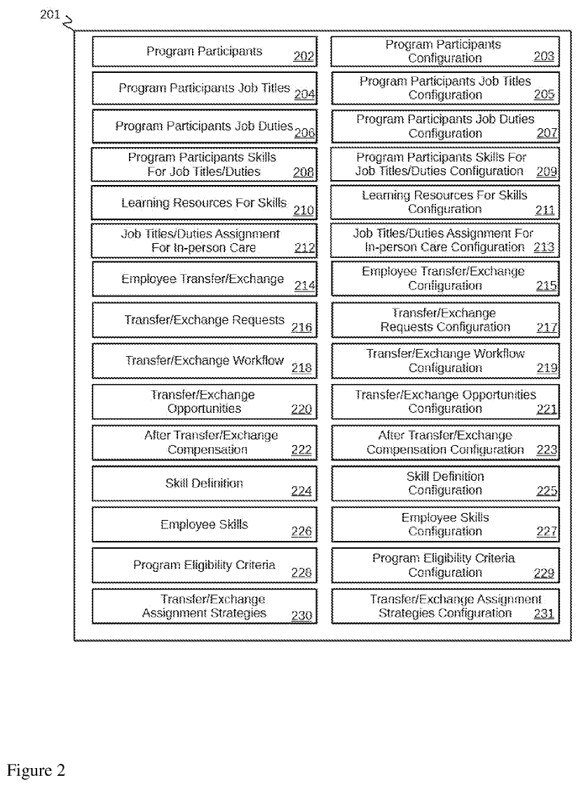

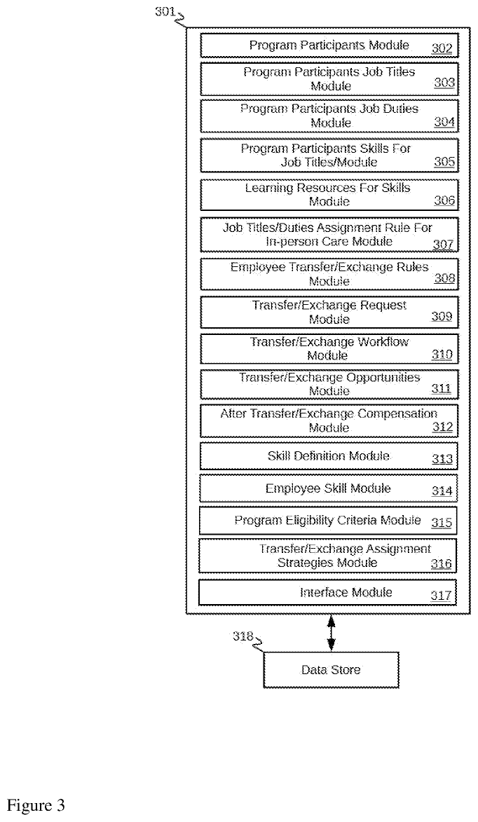

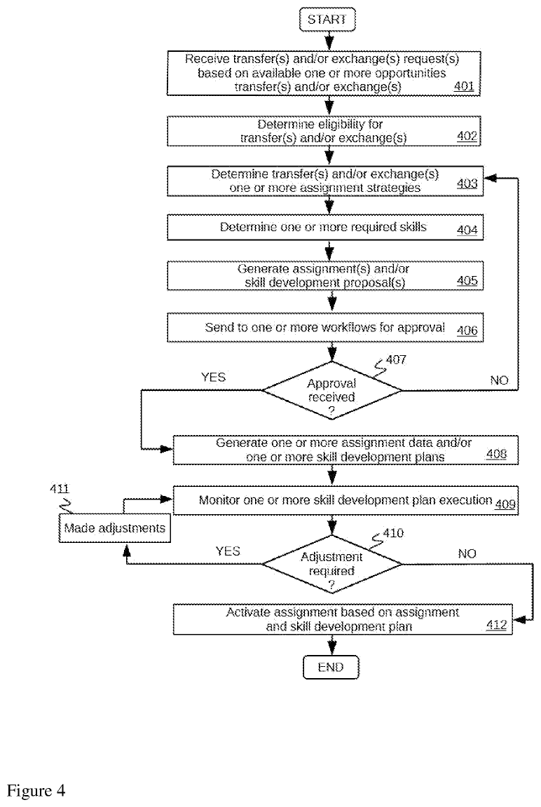

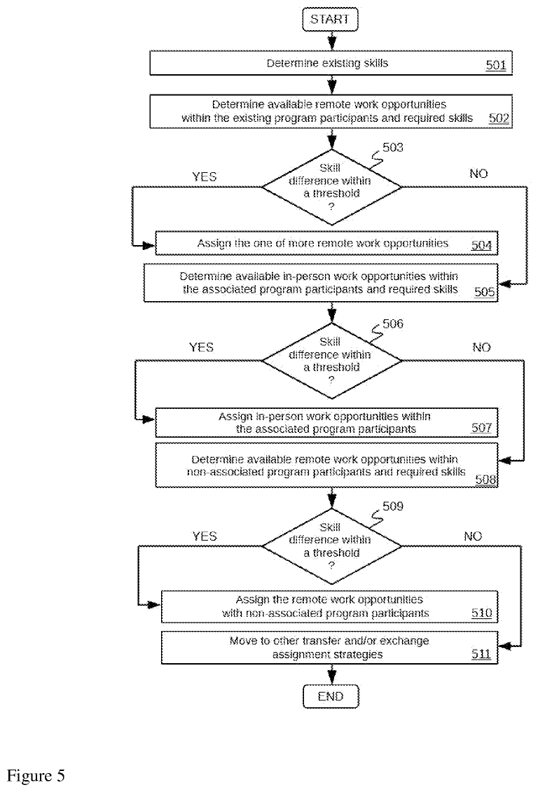

FIG. 1 shows a block diagram of an example computer 101 used to provide computing functionalities to implement the present invention. Computing devices such as laptop, workstation, laptops, distributed computing systems, desktop, server, cluster, virtual machine, mainframe, smart phone, wireless data port, a personal digital assistance, tablet computing devices are examples of such computer 101. The computer 101 includes a Processor 102 (e.g., a central processing unit (CPU), microprocessor, a digital signal processor (DSP), a conventional processor, a virtual process, micro-controller, virtual machine, a graphic processing unit (GPU), a radio-frequency integrated circuit (RFIC), an application specific integrated circuit (ASIC) or any suitable combination thereof). Processor 102 can be a multi-core processor or a plurality of multi-core processors. Memory 103 can be any kind of one or more memory devices such as read only memory (ROM), random access memory (RAM), optical, magnetic and flash memory. In some implementation, Memory 103 can be combination of two or more different types of memory. Memory 103 is shown as integral part of the computer 101, in alternative implementation memory 103 can be external to computer 101. Storage device 104 can be any medium which can be used as persistence storage. For example, hard drive, tape drive, optical disk drive, USB, flash driver and disk arrays. The storage device 104 is not limited to a particular storage device and may include memory devices such as ROM, RAM, hard disk, and the like. The storage device 104 may be a cloud storage device. Input device 105 is used to input external data and can be any kind of device such as mouse, trackball, light pen, bio-metric mechanism including voice recognition. Output device 106 can be any kind of device used for data output. For example, cathode-ray-tube (CRT) monitor, plasma display, crystal display, projector, printer and speaker. Communication interface 107 can be one or more interfaces to any kind of networks such as internet, intranet, local area network, wide area network, a telephone network such as Public Switched Telephone Network, or combination of different kinds of networks. In some embodiments, the storage device 104 may connect to the example computer 101 using communication interface 107. The example computer system 101 can be a virtual machine or a cloud based computing device. The expression “or” is not exclusive throughout the specification.

FIG. 2 illustrates an exemplary data model 201 used by the system and the computer-implemented method. A Program Participant is one or more organizations which provide one or more products and/or one or more services using one or more persons. The expression “organization” here may also refer to one or more organizational units. One or more processes which facilitate one or more in-person cares of one or more parents by using system 301 is referred as “Program”. A Program may ideally have multiple Program Participants. An employee is a person who is associated with one or more Program Participants as an employee and/or as a contractor. An elder parent is a person who is parent or grandparent of an employee through blood or marriage and needs one or more in-person cares. The expression “elder parent” also refers to parent or grandparent who are not of old age but need one or more in-person cares due to one or more medical reasons and/or one or more disabilities.

The data model Program Participants 202 stores data related to one or more program participants. The data model Program Participants Configuration 203 may store data such as one or more data schema, constraints, filters, insights, default values, validations, types, formats information, presentation templates and other configuration data which is used to create, read, delete and update data stored in the data model Program Participants 202. The data model Program Participants 202 may also store one or more rules which may be based on one or more self-learning algorithms.

The data model Program Participants Job Titles 204 stores data related to one or more job titles in use by the one or more program participants. The data model Program Participants Job Titles Configuration 205 may store data such as one or more data schema, constraints, filters, insights, default values, validations, types, formats information, presentation templates and other configuration data which is used to create, read, delete and update data stored in the data model Program Participants Job Titles 204. The data model Program Participants Job Titles 204 may also store one or more rules which may be based on one or more self-learning algorithms.

The data model Program Participants Job Duties 206 stores data related to one or more job duties associated with the one or more job titles used by the one or more program participants. The data model Program Participants Job Duties Configuration 207 may store data such as one or more data schema, constraints, filters, insights, default values, validations, types, formats information, presentation templates and other configuration data which is used to create, read, delete and update data stored in the data model Program Participants Job Duties 206. The data model Program Participants Job Duties 206 may also store one or more rules which may be based on one or more self-learning algorithms.

The data model Program Participants Skills For Job Titles/Duties 208 stores data related to one or more skills that are required for the one or more job titles and/or one or more job duties used by the one or more Program Participants. The data model Program Participants Skills For Job Titles/Duties 208 may also contain data related to one or more skill development plans and data related to monitoring of execution of the one or more skill development plans. The data model Program Participants Skills For Job Titles/Duties Configuration 209 may store data such as one or more data schema, constraints, filters, insights, default values, validations, types, formats information, presentation templates and other configuration data which is used to create, read, delete and update data stored in the data model Program Participants Skills For Job Titles/Duties 208. The data model Program Participants Skills For Job Titles/Duties 208 may also store one or more rules which may be based on one or more self-learning algorithms.

The data model Learning Resources For Skills 210 stores data related to one or more learning resources that are required to gain/upgrade the one or more skills that are required for the one or more job duties associated with the one or more job titles used by the one or more program participants. The data model Learning Resources For Skills Configuration 211 may store data such as one or more data schema, constraints, filters, insights, default values, validations, types, formats information, presentation templates and other configuration data which is used to create, read, delete and update data stored in the data model Learning Resources For Skills 210. The data model Learning Resources For Skills 210 may also store one or more rules which may be based on one or more self-learning algorithms.

The data model Job Titles/Duties Assignment For In-person Care 212 stores data related to assignment of one or more job titles and/or the one or more duties to the one or more employees associated with one or more program participants, who need to provide one or more in-person cares to their one or more elder parents. The data model Job Titles/Duties Assignment For In-person Care Configuration 213 may store data such as one or more data schema, constraints, filters, insights, default values, validations, types, formats information, presentation templates and other configuration data which is used to create, read, delete and update data stored in the data model Job Titles/Duties Assignment For In-person Care 212. The Job Titles/Duties Assignment For In-person Care 212 may also store one or more rules which may be based on one or more self-learning algorithms.

The data model Employee Transfer/Exchange 214 stores data related to one or more transfers and/or one or more exchanges of one or more employees associated with one or more program participants who need to provide one or more in-person cares to their one or more elder parents. The data model Employee Transfer/Exchange 214 may store one or more rules which may be based on one or more self-learning algorithms. The term “transfer” means one way one or more movements of one or more employees from one Program Participant to other one or more Participant Participants to enable the one or more employees to provide one or more in-person cares to their one or more elder parents. The term “exchange” refers to a case a group of Program Participants where the each Program Participant receives and transfers one or more employees to enable the one or more employees provide one or more in-person cares to their one or more elder parents. The data model Employee Transfer/Exchange Configuration 215 may store data such as one or more data schema, constraints, filters, insights, default values, validations, types, formats information, presentation templates and other configuration data which is used to create, read, delete and update data stored in the data model Employee Transfer/Exchange 214.

The data model Transfer/Exchange Requests 216 stores data related to requests by the one or more employees who need to provide one or more in-person cares to their one or more elder parents for one or more transfers and/or one or more exchanges. The data model Transfer/Exchange Requests Configuration 217 may store data such as one or more data schema, constraints, filters, insights, default values, validations, types, formats information, presentation templates and other configuration data which is used to create, read, delete and update data stored in the data model Transfer/Exchange Requests 216. The data model Transfer/Exchange Requests 216 may also store one or more rules which may be based on one or more self-learning algorithms.

The data model Transfer/Exchange Workflow 218 stores data related to one or more approval workflows and/or one or more rejection workflows for one or more transfers and/or one or more exchanges. The data model Transfer/Exchange Workflow Configuration 219 may store data such as one or more data schema, constraints, filters, insights, default values, validations, types, formats information, presentation templates and other configuration data which is used to create, read, delete and update data stored in the data model Transfer/Exchange Workflow 219. data model Transfer/Exchange Requests 216. The data model Transfer/Exchange Workflow 218 may also store one or more rules which may be based on one or more self-learning algorithms.

The data model Transfer/Exchange Opportunities 220 stores data related to one or more transfers and/or one or more exchange opportunities. The data model Transfer/Exchange Opportunities Configuration 221 may store data such as one or more data schema, constraints, filters, insights, default values, validations, types, formats information, presentation templates and other configuration data which is used to create, read, delete and update data stored in the data model Transfer/Exchange Opportunities 220. The data model Transfer/Exchange Opportunities 220 may also store one or more rules which may be based on one or more self-learning algorithms.

The data model After Transfer/Exchange Compensation 222 stores data related to one or more compensations after one or more transfers and/or one or more exchanges. The data model After Transfer/Exchange Compensation Configuration 223 may store data such as one or more data schema, constraints, filters, insights, default values, validations, types, formats information, presentation templates and other configuration data which is used to create, read, delete and update data stored in the data model After Transfer/Exchange Compensation 222. The data model After Transfer/Exchange Compensation 222 may also store one or more rules which may be based on one or more self-learning algorithms.

The data model Skill Definition 224 stores data related to one or more skills. The data model Skill Definition Configuration 225 may store data such as one or more data schema, constraints, filters, insights, default values, validations, types, formats information, presentation templates and other configuration data which is used to create, read, delete and update data stored in the data model Skill Definition 224. The data model Skill Definition 224 may also store one or more rules which may be based on one or more self-learning algorithms.

The data model Employee Skills 226 stores data related to one or more skills possessed by the one or more employees. The data model Employee Skills Configuration 227 may store data such as one or more data schema, constraints, filters, insights, default values, validations, types, formats information, presentation templates and other configuration data which is used to create, read, delete and update data stored in the data model Employee Skills 226. The data model Employee Skills 226 may also store one or more rules which may be based on one or more self-learning algorithms.

The data model Program Eligibility Criteria 228 stores data related to one or more eligibility criteria for utilizing the program. The data model Program Eligibility Criteria Configuration 229 may store data such as one or more data schema, constraints, filters, insights, default values, validations, types, formats information, presentation templates and other configuration data which is used to create, read, delete and update data stored in the data model Program Eligibility Criteria 228. The data model Program Eligibility Criteria 228 may also store one or more rules which may be based on one or more self-learning algorithms.

The data model Transfer/Exchange Strategies 230 stores data related to one or more flows of one or more steps and/or one or more acts used in determining one or more transfers and/or one or more exchanges of one or more employees who need to provide one or more in-person cares to their one or more elder parents. The data model Transfer/Exchange Strategies Configuration 231 may store data such as one or more data schema, constraints, filters, insights, default values, validations, types, formats information, presentation templates and other configuration data which is used to create, read, delete and update data stored in the data model Transfer/Exchange Strategies 230. The data model Transfer/Exchange Strategies may also store one or more rules which may be based on one or more self-learning algorithms.

It is a well known art that the data models can be merged, split and given a different name without changing their purpose to what is presented here. Such variation are within the scope of the invention.

FIG. 3 is a diagram conceptually illustrating an exemplary system 301 according to the present invention. The exemplary system 301 stores information in data store 318. The data store 318 can be one or more distributed data stores. The data store 318 may store data partially or fully in one or more clouds. The exemplary system 301 generally consists of multiple modules 302-317. It is a known art to a person skilled in the art that the modules can be split, merged and renamed without changing their purpose. All such variations are within scope of the invention. It is a known art to a person skilled in the art that a ‘module’ can be replaced with other means of grouping relevant computer instructions such as in form of function, subroutine, routine, component, services and microservices. All such variations known to a person skilled in the art are within the scope of the present invention. It is also a known art that modules can be distributed on multiple computing environments. Such variations known to a person skilled in the art are within the scope of the present inventions.

The module Program Participants Module 302 contains computer instructions to create, read, update and delete data related to the one or more program participants by mainly using the data model Program Participants 202 and the data model Program Participants Configuration 203. The Program Participants Module 302 may further contain computer instructions to perform one or more acts of by optionally using one or more stored rules in the data model Program Participants 202:
Classifying one or more program participants into one or more categories, wherein the one or more categories may be associated with one or more category hierarchies.
Classifying one or more program participants into one or more domains, wherein the one or more domains may be associated with one or more domain hierarchies.
Associating the one or more categories of the one or more program participants to the one or more domains of the one or more program participants.
Identifying one or more prospect program participants.
Ranking the one or more program participants.

The module Program Participants Job Titles Module 303 contains computer instructions to create, read, update and delete data related to the one or more program participants job titles by mainly using the data model Program Participants Job Titles 204 and the data model Program Participants Job Titles Configuration 205. The module Program Participants Job Titles Module 303 may further perform act of, by optionally using one or more stored rules in the data model Program Participants Job Titles 204:
Managing one or more relationships and/or one or more relationship strengths among one or more job titles associated with one program participant to one or more job titles associated with another program participant.

The module Program Participants Job Duties Module 304 contains computer instructions to create, read, update and delete data related to one or more program participants job duties by mainly using the data model Program Participants Job Duties 206 and the data model Program Participants Job Duties Configuration 207. The module Program Participants Job Duties Module 304 may further perform act of, by optionally using one or more stored rules in the data model Program Participants Job Duties 206:
Managing one or more relationships and/or one or more relationship strengths among one or more job duties associated with one program participant to one or more job duties associated with another program participant.

The module Program Participants Skills For Job Titles/Duties Module 305 contains computer instructions to create, read, update and delete data related to one or more program participants skills by mainly using the data model Program Participants Skills For Job Titles/Duties 208 and the data model Program Participants Skills For Job Titles/Duties Configuration 209. The module Program Participants Skills For Job Titles/Duties Module 305 may further perform act of, by optionally using one or more stored rules in the data model Program Participants Skills For Job Titles/Duties 208:
Managing one or more relationships and/or one or more relationship strengths among one or more required skills for one or more duties associated with one program participant to one or more required skills for one or more duties associated with another program participant.
Managing one or more relationships and/or one or more relationship strengths among one or more required skills for one or more titles associated with one program participant to one or more required skills for one or more titles associated with another program participant.
Determining difference among skills.
Generating proposal for one or more skill development plans.
Generating one or more skill development plans.
Assigning one or more skill development plans.
Monitoring execution of one or more skill development plans.
Adjusting one or more skill development plans.

The module Learning Resources For Skills Module 306 contains computer instructions to create, read, update and delete data related to one or more learning resources for skills by mainly using the data model Learning Resources For Skills 210 and the data model Learning Resources For Skills Configuration 211. The module Learning Resources For Skills Module 306 may perform act of, by optionally using one or more stored rules in the data model Learning Resources For Skills 210:
Managing one or more relationships and/or one or more relationship strengths among one or more learning resources for gaining/upgrading one or more skills associated with one program participant to one or more learning resources for gaining/upgrading one or more skills associated with another program participant.

The module Job Titles/Duties Assignment For In-person Care Module 307 contains computer instructions to create, read, update and delete data related to one or more job titles assignment rules and/or one or more duties assignment rules for one or more in-person parent cares for one or more employees associated with one or more Program Participants by mainly using the data model Job Titles/Duties Assignment For In-person Care 212 and the data model Job Titles/Duties Assignment For In-person Care Configuration 213. The Job Titles/Duties Assignment For In-person Care Module 307 may perform act of, by optionally using one or more stored rules in the data model Job Titles/Duties Assignment For In-person Care 212:
Managing one or more relationships and/or one or more relationship strengths, among one or more job titles assignment rules and/or one or more duties assignment rules associated with one program participant, to one or more job titles assignment rules and/or one or more duties assignment rules associated with another program participant.

The module Employee Transfer/Exchange Module 308 contains computer instructions to create, read, update and delete data related to one or more employee exchanges and/or one or more employee transfer mainly using the data model Employee Transfer/Exchange 214 and the data model Employee Transfer/Exchange Configuration 215. The module Employee Transfer/Exchange Module 308 may perform acts of, by optionally using one or more stored rules in the data model Employee Transfer/Exchange 214:
Determining eligibility for one or more transfers for one or more employees associated with one or more program participants.
Determining further eligibility for one or more transfers for one or more employees associated with one or more program participants based on one or more aspects of:
   protecting one or more trade secrets,
   protecting one or more intellectual properties,
   protecting one or more advantages over one or more competitors,
   one or more duties health requirements,
   one or more criminal histories,
   one or more work authorizations.
Determining eligibility for one or more exchange for one or more employees associated with one or more program participants.
Determining further eligibility for one or more exchanges for one or more employees associated with one or more program participants based on one or more aspects of:
   protecting one or more trade secrets,
   protecting one or more intellectual properties,
   protecting one or more advantages over one or more competitors,
   one or more duties health requirements,
   one or more criminal histories,
   one or more work authorizations.
Generating one or more assignments to one or more transfers and/or one or more exchanges for one or more employees associated with one or more program participants.
Activating one or more assignments to one or more transfers and/or one or more exchanges for one or more employees associated with one or more program participants.

The module Transfer/Exchange Request Module 309 contains computer instructions to create, read, update and delete data related to requests for one or more transfers and/or one or more exchanges by mainly using the data model Transfer/Exchange Requests 216 and the data model Transfer/Exchange Requests Configuration 217. The module Transfer/Exchange Request Module 309 may further contain computer instructions to perform one or more acts of, by optionally using one or more stored rules in the data model Transfer/Exchange Requests 216:
Classifying one or more transfer requests and/or one or more exchange requests into one or more categories, wherein the one or more categories may be associated with one or more category hierarchies.
Classifying one or more transfer requests and/or one or more exchange requests into one or more domains, wherein the one or more domains may be associated with one or more domain hierarchies.
Associating the one or more categories of the one or more transfer requests and/or one or more exchange requests to the one or more domains of the one or more one or more transfer requests and/or one or more exchange requests.
Ranking the one or more one or more transfer requests and/or one or more exchange requests.
Receive one or more transfer requests and/or one or more exchange requests based on Transfer/Exchange Opportunities 220.

The module Transfer/Exchange Workflow Module 310 contains computer instructions to create, read, update and delete data related to one or more workflows for one or more transfers and/or one or more exchanges by mainly using the data model Transfer/Exchange Workflow 218 and the data model Transfer/Exchange Workflow Configuration 219. The module Transfer/Exchange Workflow Module 310 may further contain computer instructions to perform one or more acts of, by optionally using one or more stored rules in the data model Transfer/Exchange Workflow 218:
Determining one or more relevant workflows.
Determining responsible one or more persons for the determined one or more relevant workflows.

The module Transfer/Exchange Opportunities Module 311 contains computer instructions to create, read, update and delete data related to one or more opportunities for one or more transfers and/or one or more exchanges by mainly using the data model Transfer/Exchange Opportunities 220 and the data model Transfer/Exchange Opportunities Configuration 221. Transfer/Exchange Opportunities Module 311 may further contain computer instructions to perform one or more acts of, by optionally using one or more stored rules in the data model Transfer/Exchange Opportunities 220:
Classifying one or more transfer opportunities and/or one or more exchange opportunities into one or more categories, wherein the one or more categories may be associated with one or more category hierarchies.
Classifying one or more transfer opportunities and/or one or more exchange opportunities into one or more domains, wherein the one or more domains may be associated with one or more domain hierarchies.
Associating the one or more categories of the one or more transfer opportunities and/or one or more exchange opportunities to the one or more domains of the one or more one or more transfer requests and/or one or more exchange requests.
Ranking the one or more one or more transfer opportunities and/or one or more exchange opportunities.

The module After Transfer/Exchange Compensation Module 312 contains computer instructions to create, read, update and delete data related to one or more compensations after one and more transfers and one or more exchanges by mainly using the data model After Transfer/Exchange Compensation 222 and the data model After Transfer/Exchange Compensation Configuration 223. The module After Transfer/Exchange Compensation Module 312 may further contain computer instructions to perform one or more acts of, by optionally using one or more stored rules in the data model After Transfer/Exchange Compensation 222:
Classifying one or more after transfer compensations and/or one or more after exchange compensations into one or more categories, wherein the one or more categories may be associated with one or more category hierarchies.
Classifying one or more after exchange compensations and/or one or more after exchange compensations into one or more domains, wherein the one or more domains may be associated with one or more domain hierarchies.
Associating the one or more categories of the one or more after transfer compensations and/or one or more after exchange compensations to the one or more domains of the one or more one or more after transfer compensations and/or one or more after exchange compensations.
Ranking the one or more one or more after transfer compensations and/or one or more after exchange compensations.

The module Skill Definition Module 313 contains computer instructions to create, read, update and delete data related to definition of one or more skills by mainly using the data model Skill Definition 224 and the data model Skill Definition Configuration 225. The module Skill Definition Module 313 may further contain computer instructions to perform one or more acts of by optionally using one or more stored rules in the data model Skill Definition 224:
Classifying one or more skills into one or more categories, wherein the one or more categories may be associated with one or more category hierarchies.
Classifying one or more skills into one or more domains, wherein the one or more domains may be associated with one or more domain hierarchies.
Associating the one or more categories of the one or more skills to the one or more domains of the one or more skills.
Identifying one or more prospect skills.
Ranking the one or more skills.

The module Employee Skill Module 314 contains computer instructions to create, read, update and delete data related to one or more skills possessed by one or more employees by mainly using the data model Employee Skills 226 and the data model Employee Skills Configuration 227. The module Employee Skill Module 314 may further contain computer instructions to perform one or more acts of by optionally using one or more stored rules in the data model Employee Skills 226:
Classifying one or more employee skills into one or more categories, wherein the one or more categories may be associated with one or more category hierarchies.
Classifying one or more employee skills into one or more domains, wherein the one or more domains may be associated with one or more domain hierarchies.
Associating the one or more categories of the one or more employee skills to the one or more domains of the one or more employee skills.
Identifying one or more prospect employee skills.
Ranking the one or more employee skills.

The module Program Eligibility Criteria Module 315 contains computer instructions to create, read, update and delete data related to one or more eligibility criteria for the one or more programs by mainly using the data model Program Eligibility Criteria 228 and the data model Program Eligibility Criteria Configuration 229. The module Program Eligibility Criteria Module 315 may perform act of, by optionally using one or more stored rules in the data model Program Eligibility Criteria 228:
Determining eligibility for the one or more prospect program participants and one or more program participants.

The module Transfer/Exchange Assignment Strategies Module 316 contains computer instructions to create, read, update and delete data related to one or more strategies for one or more transfers and/or one or more exchanges by mainly using the data model Transfer/Exchange Assignment Strategies 230 and the data model Transfer/Exchange Assignment Strategies Configuration 231. The module Transfer/Exchange Assignment Strategies Module 316 may perform one or more acts of, by optionally using one or more stored rules in the data model Transfer/Exchange Assignment Strategies 230:
Determining one or more assignment strategies for one or more transfers for one or more employees associated with one or more program participants.
Determining one or more assignment strategies for one or more exchanges for one or more employees associated with one or more program participants.

The module Interface Module 317 contains computer instructions to provide one or more interfaces to one or more modules 302-316 with or without adding further processing. The module Interface Module 317 may also enable interaction with the system 301 through one or more graphical user interfaces such as use of one or more portals, one or more mobile apps running on one or more mobile phones, tablets and laptops. The module Interface Module 317 may further enable interaction with the system 301 through one or more APIs. The data received by the Interface Module 317 through one or more APIs may go through one or more extract transfer load (ETL) and/or extract load transfer (ELT) processes. The access to the system 301 may use one or more access control methods such as: using one or more certificates, using one or more session keys, using one or more bio-metric data, using one or more user passwords, using one or more single sign-on methods, using one or more password wallets, using one or more cookies, using one or more stored configurations and using two (plus) factors authentications.

FIG. 4 is a flow chart illustrating an exemplary operation of enabling one or more transfers and/or one or more exchanges according to the present invention. The system 301 receives one or more requests for one or more transfers and/or one or more exchanges from one or more employees who need to provide one or more in-person cares to one or more elder parents based on available one or more opportunities for one or more exchanges and/or one or more transfer (act 401). The one or more opportunities for one or more exchange and/or one or more transfers may be currently available and/or available at specific time in future. The one or more opportunities for one or more exchange and/or one or more transfers may be based on one or more requests from one or more employees to add one or more new opportunities or modify the existing opportunities. The system 301 determines eligibility for one or more transfers and/or one or more exchanges by using one or more eligibility criteria (act 402). The system 301 determines one or more transfers assignment strategies and/or one or more exchange assignment strategies (act 403) when the one or more employees are eligible for the one or more transfers and/or one or more exchanges. The system 301 determines one or more required skills after one or more transfers and/or one or more exchanges (act 404). The system generates one or more assignments to one or more transfers and/or one or more exchanges, and/or one or more skill development proposals (act 405). The system 301 sends the generated one or more assignments to one or more transfers and/or one or more exchanges, and/or one or more skill development proposals, for approval to one or more workflows (act 406). In case, the approval is received, one or more assignments data and/or one or more skill development plans are generated (act 408) (act 407). The system 301 monitors progress of the generated one or more skill development plans execution based on the received data related to the progress (act 409). The monitoring of the progress of execution may be based on stored one or more rules optionally requiring human input (act 409). The system 301 optionally makes one or more adjustments to the one or more assignments data and/or one or more skill development plans (act 410 and act 411). The one or more adjustments may be based on one or more stored rules optionally requiring human input (act 411). The system 301 activates one or more assignments data according to the one or more assignments and/or one or more skill development plans (act 412). The activation may be based on one or more stored rules optionally requiring human input (act 412). The flow of FIG. 4 may calculate one or more compensations for the one or more assignments.

FIG. 5 is a flow chart illustrating an exemplary one or more transfers and one or more exchange strategies according to the present invention. The system 301 determines existing one or more skills of the one or more employees who are requesting one or more transfers and/or one or more exchanges to provide one or more in-person cares to one or more elder parents (act 501). The system 301 determines available one or more remote work opportunities within the one or more Program Participants associated with the one or more employees and the required one or more skills (act 502). When the difference between one or more skills possessed by the one or more employees requesting one or more transfers and/or one or more exchanges, and one or more skills required for the available one or more remote work opportunities within the one or more Program Participants associated with the one or more employees, is within a threshold, the one or more requesting employees are assigned to one or more of internal remote work opportunities after approval workflow (act 503) (act 504). When the skill difference possessed by the one or more employees requesting one or more transfers and/or one or more exchanges, and one or more skills required for the available one or more remote work opportunities within the one or more Program Participants associated with the one or more employees, is above a threshold, the system 301 determines available one or more in-person work opportunities within the one or more Program Participants associated with the one or more employees and the required one or more skills which can enable the one or more employees provide one or more in-person cares to their one or more elder parents (act 503) (act 505). When the skill difference possessed by the one or more employees requesting one or more transfers and/or one or more exchanges, and one or more skills required for the available one or more in-person work opportunities is within a threshold, the one or more in-person work opportunities within the one or more Program Participants associated with the one or more employees, are assigned to the requesting one or more employees after approval workflow (act 506) (act 507). When the skill difference possessed by the one or more employees requesting one or more transfers and/or one or more exchanges, and the one or more skills required for the available one or more in-person work opportunities within the one or more Program Participants associated with the one or more employees, is beyond a threshold, the system 301 determines available one or more remote work opportunities in other one or more Program Participants which are not associated with the one or more employees and the required one or more skills which can enable the one or more employees provide one or more in-person cares to their one or more elder parents (act 506)(act 508). When the skill difference possessed by the one or more employees requesting one or more transfers and/or one or more exchanges, and skills required for the available one or more remote work opportunities in one or more Program Participants which are not associated with the one or more employees is within a threshold, the one or more remote work opportunities in the one or more Program Participants which are not associated with the one or more employees are assigned to the requesting one or more employees after approval workflow (act 509) (act 510). When the skill difference possessed by the one or more employees requesting one or more transfers and/or one or more exchanges, and one or more skills required for the available one or more remote work opportunities in the one or more Program Participants which are not associated with the one or more employees, is above a threshold, the system 301 explores one or more other assignment methods (act 511).

It is a well known art that acts/steps in flow chart can be split, merged and reordered. Such variations to flow in FIG. 4 is within scope of the invention.

It is to be understood that while the detailed description describes the present invention, the foregoing description is for illustrative purpose and does not limit the scope of the present invention which is defined by the scope of the appended claims. Other embodiments, arrangements and equivalents will be evident to those skilled in the art. Such other embodiments, arrangements and equivalents are within the scope of the present invention as defined by the appended claims.

1. A system to facilitate in-person cares of elder parents by employees, the system comprising:
one or more processors coupled to one or more memories;
stored one or more program participants data and related stored configuration data;
stored one or more program participants job titles data and related stored configuration data;
stored one or more program participants job duties data and related stored configuration data;
stored one or more program participants skills for job titles data and related stored configuration data;
stored one or more program participants skills for job duties data and related stored configuration data;
stored one or more learning resources for skills data and related stored configuration data;
stored one or more job titles assignment rules data and related stored configuration data;
stored one or more job duties assignment rules data and related stored configuration data;
stored one or more employee transfers data and related stored configuration data;
stored one or more employee exchanges data and related stored configuration data;
stored one or more transfer requests data and related stored configuration data;
stored one or more exchange requests data and related stored configuration data;
stored one or more transfer workflows data and related stored configuration data;
stored one or more exchange workflows data and related stored configuration data;
stored one or more transfer opportunities data and related stored configuration data;
stored one or more exchange opportunities data and related stored configuration data;
stored one or more transfer compensation data and related stored configuration data;
stored one or more exchange compensation data and related stored configuration data;
stored one or more skills definition data and related stored configuration data;
stored one or more employee skills data and related stored configuration data;
stored one or more program eligibility criteria data and related stored configuration data;
stored one or more transfer strategies data and related stored configuration data;
stored one or more exchange strategies data and related stored configuration data;
one or more computer-executable instructions configured to receive one or more requests for one or more transfers and/or one or more exchanges from one or more employees who need to provide one or more in-person cares to one or more elder parents based on available one or more opportunities for one or more exchanges and/or one or more transfers.

2. The system of claim 1, further comprising at least one of:
one or more computer-executable instructions configured to classify one or more program participants into one or more categories with or without using one or more stored rules which are optionally based on one or more self-learning algorithms and stored as the configuration data related to the stored one or more program participants data;
one or more computer-executable instructions configured to classify one or more program participants into one or more domains with or without using one or more stored rules which are optionally based on one or more self-learning algorithms and stored as the configuration data related to the stored one or more program participants data;
one or more computer-executable instructions configured to associate one or more categories of one or more program participants to one or more domains of one or more program participants with or without using one or more stored rules which are optionally based on one or more self-learning algorithms and stored as the configuration data related to the stored one or more program participants data;
one or more computer-executable instructions configured to rank one or more program participants with or without using one or more stored rules which are optionally based on one or more self-learning algorithms and stored as the configuration data related to the stored one or more program participants data;
one or more computer-executable instructions configured to identify one or more prospect program participants with or without using one or more stored rules which are optionally based on one or more self-learning algorithms and are stored as the configuration data related to the stored one or more program participants data;
the stored configuration data related to the stored one or more program participants data optionally storing one or more of:
constraints,
filters,
insights,
validations,
presentation templates.

3. The system of claim 1, further comprising at least one of:
one or more computer-executable instructions configured to manage one or more relationships and optionally one or more relationship strengths, among one or more job titles associated with one program participant, to one or more job titles associated with another program participant with or without using one or more stored rules which are optionally based on one or more self-learning algorithms and stored as the configuration data related to the stored one or more program participants job titles data;
one or more computer-executable instructions configured to manage one or more relationships and optionally one or more relationship strengths, among one or more job duties associated with one program participant, to one or more job duties associated with another program participant with or without using one or more stored rules which are stored as the configuration data related to the stored one or more program participants job duties data;
the stored configuration data related to the stored one or more program participants job titles data optionally storing one or more of:
constraints,
filters,
insights,
validations,
presentation templates;
the stored configuration data related to the stored one or more program participants job duties data optionally storing one or more of:
constraints,
filters,
insights,
validations,
presentation templates.

4. The system of claim 1, further comprising at least one of:
one or more computer-executable instructions configured to manage one or more relationships and optionally one or more relationship strengths, among one or more required skills for one or more duties associated with one program participant to one or more required skills for one or more duties associated with another program participant with or without using one or more stored rules which are optionally based on one or more self-learning algorithms and stored as the configuration data related to the stored one or more program participants skills for job duties data;
one or more computer-executable instructions configured to manage one or more relationships and optionally one or more relationship strengths, among one or more required skills for one or more titles associated with one program participant to one or more required skills for one or more titles associated with another program participant with or without using one or more stored rules which are optionally based on one or more self-learning algorithms and stored as the configuration data related to the stored one or more program participants skills for job titles data;
one or more computer-executable instructions configured to determine difference among skills with or without using one or more stored rules which are optionally based on one or more self-learning algorithms and stored as the configuration data related to the stored one or more program participants skills for job titles data or stored as the configuration data related to the stored one or more program participants skills for job duties data;
one or more computer-executable instructions configured to generate proposal for one or more skill development plans with or without using one or more stored rules which are optionally based on one or more self-learning algorithms and stored as the configuration data related to the stored one or more program participants skills for job titles data or stored as the configuration data related to the stored one or more program participants skills for job duties data;
one or more computer-executable instructions configured to generate one or more skill development plans with or without using one or more stored rules which are optionally based on one or more self-learning algorithms and stored as the configuration data related to the stored one or more program participants skills for job titles data or stored as the configuration data related to the stored one or more program participants skills for job duties data;
one or more computer-executable instructions configured to assign one or more skill development plans with or without using one or more stored rules which are optionally based on one or more self-learning algorithms and stored as the configuration data related to the stored one or more program participants skills for job titles data or stored as the configuration data related to the stored one or more program participants skills for job duties data;
the stored configuration data related to the stored one or more program participants skills for job titles data optionally storing one or more of:
constraints,
filters,
insights,
validations,
presentation templates;
the stored configuration data related to the stored one or more program participants skills for job duties data optionally storing one or more of:
constraints,
filters,
insights,
validations,
presentation templates.

5. The system of claim 1, further comprising at least one of:
one or more computer-executable instructions configured to manage one or more relationships and optionally one or more relationship strengths, among one or more learning resources for gaining or upgrading one or more skills associated with one program participant, to one or more learning resources for gaining or upgrading one or more skills associated with another program participant with or without using one or more stored rules which are optionally based on one or more self-learning algorithms and stored as the configuration data related to the stored one or more learning resources for skills data;
the stored configuration data related to the stored one or more learning resources for skills data optionally storing one or more of:
constraints,
filters,
insights,
validations,
presentation templates.

6. The system of claim 1, further comprising at least one of:
one or more computer-executable instructions configured to manage one or more relationships and optionally one or more relationship strengths, among one or more job titles assignment rules associated with one program participant, to one or more job titles assignment rules associated with another program participant;
one or more computer-executable instructions configured to manage one or more relationships and optionally one or more relationship strengths, among one or more job duties assignment rules associated with one program participant, to one or more job duties assignment rules associated with another program participant;
the stored configuration data related to the stored job titles assignment rules data optionally storing one or more of:
constraints,
filters,
insights,
validations,
presentation templates;
the stored configuration data related to the stored one or more job duties assignment rules data optionally storing one or more of:
constraints,
filters,
insights,
validations,
presentation templates.

7. The system of claim 1, further comprising at least one of:
one or more computer-executable instructions configured to determine eligibility, with or without using one or more stored rules which are optionally based on one or more self-learning algorithms and stored as the configuration data related to the stored one or more employee transfers data, for the one or more transfers for the one or more employees associated with one or more program participants optionally based on one or more aspects of:
protecting one or more trade secrets,
protecting one or more intellectual properties,
protecting one or more advantages over one or more competitors,
one or more duties health requirements,
one or more criminal histories,
one or more work authorizations;
one or more computer-executable instructions configured to determine eligibility, with or without using one or more stored rules which are optionally based on one or more self-learning algorithms and stored as the configuration data related to the stored one or more employee exchanges data, for the one or more exchanges for the one or more employees associated with one or more program participants optionally based on one or more aspects of:
protecting one or more trade secrets,
protecting one or more intellectual properties,
protecting one or more advantages over one or more competitors,
one or more duties health requirements,
one or more criminal histories,
one or more work authorizations;
one or more computer-executable instructions configured to generate one or more assignments to the one or more transfers for the one or more employees associated with one or more program participants with or without using one or more stored rules which are optionally based on one or more self-learning algorithms and stored as the configuration data related to the stored one or more employee transfers data;
one or more computer-executable instructions configured to generate one or more assignments to the one or more exchanges for the one or more employees associated with one or more program participants with or without using one or more stored rules which are optionally based on one or more self-learning algorithms and stored as the configuration data related to the stored one or more employee exchanges data;
one or more computer-executable instructions configured to activate one or more assignments to the one or more transfers for the one or more employees associated with one or more program participants with or without using one or more stored rules which are optionally based on one or more self-learning algorithms and stored as the configuration data related to the stored one or more employee transfers data;
one or more computer-executable instructions configured to activate one or more assignments to the one or more exchanges for the one or more employees associated with one or more program participants with or without using one or more stored rules which are optionally based on one or more self-learning algorithms and stored as the configuration data related to the stored one or more employee exchanges data;
the stored configuration data related to the stored one or more employee transfers data optionally storing one or more of:
constraints,
filters,
insights,
validations,
presentation templates;
the stored configuration data related to the stored one or more employee exchanges data optionally storing one or more of:
constraints,
filters,
insights,
validations,
presentation templates;

8. The system of claim 1, further comprising at least one of:
one or more computer-executable instructions configured to classify the one or more transfer requests into one or more categories with or without using one or more stored rules which are optionally based on one or more self-learning algorithms and stored as the configuration data related to the stored one or more transfer request data;
one or more computer-executable instructions configured to classify the one or more exchange requests into one or more categories with or without using one or more stored rules which are optionally based on one or more self-learning algorithms and stored as the configuration data related to the stored one or more exchange request data;
one or more computer-executable instructions configured to classify the one or more transfer requests into one or more domains with or without using one or more stored rules which are optionally based on one or more self-learning algorithms and stored as the configuration data related to the stored one or more transfers request data;
one or more computer-executable instructions configured to classify the one or more exchange requests into one or more domains with or without using one or more stored rules which are optionally based on one or more self-learning algorithms and stored as the configuration data related to the stored one or more exchange requests data;
one or more computer-executable instructions configured to associate one or more categories of the one or more transfer requests to one or more domains of the one or more transfer requests with or without using one or more stored rules which are optionally based on one or more self-learning algorithms and stored as the configuration data related to the stored one or more transfer request data;
one or more computer-executable instructions configured to associate one or more categories of the one or more exchange requests to one or more domains of the one or more exchange requests with or without using one or more stored rules which are optionally based on one or more self-learning algorithms and stored as the configuration data related to the stored one or more exchange requests data;
one or more computer-executable instructions configured to rank the one or more transfer requests with or without using one or more stored rules which are optionally based on one or more self-learning algorithms and stored as the configuration data related to the stored one or more transfer request data;
one or more computer-executable instructions configured to rank the one or more exchange requests with or without using one or more stored rules which are optionally based on one or more self-learning algorithms and stored as the configuration data related to the stored one or more exchange requests data;
one or more computer-executable instructions configured to receive the one or more transfer requests based on transfer opportunities with or without using one or more stored rules which are optionally based on one or more self-learning algorithms and stored as the configuration data related to the stored one or more transfer request data;
one or more computer-executable instructions configured to receive the one or more exchange requests based on exchange opportunities with or without using one or more stored rules which are optionally based on one or more self-learning algorithms and stored as the configuration data related to the stored one or more exchange request data;
the stored configuration data related to the stored one or more transfers data, optionally storing one or more of:
constraints,
filters,
insights,
validations,
presentation templates;
the stored configuration data related to the stored one or more exchanges data, optionally storing one or more of:
constraints,
filters,
insights,
validations,
presentation templates.

9. The system of claim 1, further comprising at least one of:
one or more computer-executable instructions configured to determine one or more relevant workflows with or without using one or more stored rules which are optionally based on one or more self-learning algorithms and stored as the configuration data related to the stored one or more transfer workflows data;
one or more computer-executable instructions configured to determine one or more relevant workflows with or without using one or more stored rules which are optionally based on one or more self-learning algorithms and stored as the configuration data related to the stored one or more exchange workflows data;
one or more computer-executable instructions configured to determine responsible one or more persons for the determined one or more relevant workflows with or without using one or more stored rules which are optionally based on one or more self-learning algorithms and stored as the configuration data related to the stored one or more transfer workflows data;
one or more computer-executable instructions configured to determine responsible one or more persons for the determined one or more relevant workflows with or without using one or more stored rules which are optionally based on one or more self-learning algorithms and stored as the configuration data related to the stored one or more exchange request data;
the stored configuration data related to the stored one or more workflows for the one or more transfers optionally storing one or more of:
constraints,
filters,
insights,
validations,
presentation templates;
the stored configuration data related to the stored one or more workflows for one or more exchanges data optionally storing one or more of:
constraints,
filters,
insights,
validations,
presentation templates.

10. The system of claim 1, further comprising at least one of:
one or more computer-executable instructions configured to classify the one or more transfer opportunities into one or more categories with or without using one or more stored rules which are optionally based on one or more self-learning algorithms and stored as the configuration data related to the stored one or more transfer opportunities data;
one or more computer-executable instructions configured to classify the one or more exchange opportunities into one or more categories with or without using one or more stored rules which are optionally based on one or more self-learning algorithms and stored as the configuration data related to the stored one or more exchange opportunities data;
one or more computer-executable instructions configured to classify the one or more transfer opportunities into one or more domains with or without using one or more stored rules which are optionally based on one or more self-learning algorithms and stored as the configuration data related to the stored one or more transfer opportunities data;
one or more computer-executable instructions configured to classify the one or more exchange opportunities into one or more domains with or without using one or more stored rules which are optionally based on one or more self-learning algorithms and stored as the configuration data related to the stored one or more exchange opportunities data;
one or more computer-executable instructions configured to associate one or more categories of the one or more transfer opportunities to one or more domains of the one or more transfer opportunities with or without using one or more stored rules which are optionally based on one or more self-learning algorithms and stored as the configuration data related to the stored one or more transfer opportunities data;
one or more computer-executable instructions configured to associate one or more categories of the one or more exchange opportunities to one or more domains of the one or more exchange opportunities with or without using one or more stored rules which are optionally based on one or more self-learning algorithms and stored as the configuration data related to the stored one or more exchange opportunities data;
one or more computer-executable instructions configured to rank one or more the one or more transfer opportunities with or without using one or more stored rules which are optionally based on one or more self-learning algorithms and stored as the configuration data related to the stored one or more transfer opportunities data;
one or more computer-executable instructions configured to rank the one or more exchange opportunities with or without using one or more stored rules which are optionally based on one or more self-learning algorithms and stored as the configuration data related to the stored one or more exchange opportunities data;
the stored configuration data related to the stored opportunities for one or more transfers data optionally storing one or more of:
constraints,
filters,
insights,
validations,
presentation templates.
the stored configuration data related to the stored opportunities for one or more exchanges data optionally storing one or more of:
constraints,
filters,
insights,
validations,
presentation templates.

11. The system of claim 1, further comprising at least one of:
one or more computer-executable instructions configured to classify one or more after transfer compensations into one or more categories with or without using one or more stored rules which are optionally based on one or more self-learning algorithms and stored as the configuration data related to the stored one or more transfer compensation data;
one or more computer-executable instructions configured to classify one or more after exchange compensations into one or more categories with or without using one or more stored rules which are optionally based on one or more self-learning algorithms and stored as the configuration data related to the stored one or more exchange compensation data;
one or more computer-executable instructions configured to classify one or more after transfer compensations into one or more domains with or without using one or more stored rules which are optionally based on one or more self-learning algorithms and stored as the configuration data related to the stored one or more exchange compensation data;
one or more computer-executable instructions configured to classify one or more after exchange compensations into one or more domains with or without using one or more stored rules which are optionally based on one or more self-learning algorithms and stored as the configuration data related to the stored one or more transfer compensation data;
one or more computer-executable instructions configured to associate one or more categories of one or more after transfer compensations to one or more domains of one or more after transfer compensations with or without using one or more stored rules which are optionally based on one or more self-learning algorithms and stored as the configuration data related to the stored exchange compensation data;
one or more computer-executable instructions configured to associate one or more categories of one or more after exchange compensations to one or more domains of one or more after exchange compensations with or without using one or more stored rules which are optionally based on one or more self-learning algorithms and stored as the configuration data related to the stored exchange compensation data;
one or more computer-executable instructions configured to rank one or more after transfer compensations with or without using one or more stored rules which are optionally based on one or more self-learning algorithms and stored as the configuration data related to the stored one or more transfer compensation data;
one or more computer-executable instructions configured to rank one or more after exchange compensations with or without using one or more stored rules which are optionally based on one or more self-learning algorithms and stored as the configuration data related to the stored one or more exchange compensation data;
the stored configuration data related to the stored one or more compensations after one and more transfers optionally storing one or more of:
constraints,
filters,
insights,
validations,
presentation templates;
the stored configuration data related to the stored one or more exchanges data optionally storing one or more of:
constraints,
filters,
insights,
validations,
presentation templates.

12. The system of claim 1, further comprising at least one of:
one or more computer-executable instructions configured to classify one or more employee skills into one or more categories with or without using one or more stored rules which are optionally based on one or more self-learning algorithms and stored as the configuration data related to the stored one or more employee skills data;
one or more computer-executable instructions configured to classify one or more employee skills into one or more domains with or without using one or more stored rules which are optionally based on one or more self-learning algorithms and stored as the configuration data related to the stored one or more employee skills data;
one or more computer-executable instructions configured to associate one or more categories of one or more employee skills to one or more domains of one or more employee skills with or without using one or more stored rules which are optionally based on one or more self-learning algorithms and stored as the configuration data related to the stored one or more employee skills data;
one or more computer-executable instructions configured to rank one or more employee skills with or without using one or more stored rules which are optionally based on one or more self-learning algorithms and stored as the configuration data related to the stored one or more employee skills data;
one or more computer-executable instructions configured to identify one or more prospect employee skills with or without using one or more stored rules which are optionally based on one or more self-learning algorithms and stored as the configuration data related to the stored one or more employee skills data;
the stored configuration data related to the stored one or more skills definition data optionally storing one or more of:
constraints,
filters,
insights,
validations,
presentation templates.

13. The system of claim 1, further comprising at least one of:
one or more computer-executable instructions configured to determine eligibility for one or more prospect program participants and one or more program participants with or without using one or more stored rules which are optionally based on one or more self-learning algorithms and stored as the configuration data related to the stored one or more program eligibility criteria data;
the stored configuration data related to the stored one or more skills possessed by the one or more employees optionally storing one or more of:
constraints,
filters,
insights,
validations,
presentation templates.

14. The system of claim 1, further comprising at least one of:
one or more computer-executable instructions configured to determine one or more assignment strategies for the one or more transfers for the one or more employees associated with one or more program participants with or without using one or more stored rules which are optionally based on one or more self-learning algorithms and stored as the configuration data related to the stored one or more program transfer assignment strategies data;
one or more computer-executable instructions configured to determine one or more assignment strategies for the one or more exchanges for the one or more employees associated with one or more program participants with or without using one or more stored rules which are optionally based on one or more self-learning algorithms and stored as the configuration data related to the stored one or more program exchange assignment strategies data;
the stored configuration data related to the stored eligibility criteria for the one or more programs data optionally storing one or more of:
constraints,
filters,
insights,
validations,
presentation templates.

15. The system of claim 1, further configured to:
determine eligibility for the one or more transfers and/or the one or more exchanges by using one or more eligibility criteria;
determine one or more transfers assignment strategies and/or one or more exchange assignment strategies when the one or more employees are eligible for the one or more transfers and/or for the one or more exchanges;
determine one or more required skills after the one or more transfers and/or the one or more exchanges;
generate one or more assignments to the one or more transfers and/or the one or more exchanges, and optionally one or more skill development proposals;
send the generated one or more assignments to the one or more transfers and/or one or more the exchanges, and/or one or more skill development proposals, for approval to one or more workflows;
generate one or more assignments data and/or one or more skill development plans when the approval is received;
monitor progress of the generated one or more skill development plans execution based on received data related to the progress optionally based on stored one or more rules, with or without human input;
optionally making one or more adjustments to the one or more assignments data and/or one or more skill development plans based on one or more stored rules optionally, with or without human input;
activate one or more assignments data according to the one or more assignments and/or the one or more skill development plans optionally based on one or more stored rules, with or without human input.

16. The system of claim 1, further configured to:
determine existing one or more skills of the one or more employees who are requesting the one or more transfers and/or the one or more exchanges to provide one or more in-person cares to one or more elder parents;
determine each of:
(a) available one or more remote work opportunities within one or more program participants associated with the one or more employees and required one or more skills,
(b) available one or more in-person work opportunities within one or more program participants associated with the one or more employees and required one or more skills which can enable the one or more employees provide one or more in-person cares to their one or more elder parents,
(c) available one or more remote work opportunities in other one or more program participants which are not associated with the one or more employees and required one or more skills which can enable the one or more employees provide one or more in-person cares to their one or more elder parents;
determine skill difference between the determined existing one or more skills of the one or more employees and following (d) (e) (f) being within one or more thresholds:
(d) the required one or more skills for the available one or more remote work opportunities within one or more program participants associated with the one or more employees,
(e) the required one or more skills for the available one or more in-person work opportunities within one or more program participants associated with the one or more employees,
(f) the required one or more skills for the available one or more remote work opportunities in other one or more program participants which are not associated with the one or more employees;
after approval workflow assign to the one or more employees at least one of when the determined skill difference is within limit:
(g) the determined one or more of internal remote work opportunities,
(h) the determined one or more in-person work opportunities within the one or more program participants associated with the one or more employees,
(i) the determined one or more remote work opportunities in the one or more program participants which are not associated with the one or more employees.

17. A computer-implemented method to facilitate in-person cares of elder parents by employees, executed by one or more computing devices each of which comprising of one or more processors coupled to one or more memories, the method comprising:
stored one or more program participants data and related stored configuration data;
stored one or more program participants job titles data and related stored configuration data;
stored one or more program participants job duties data and related stored configuration data;
stored one or more program participants skills for job titles data and related stored configuration data;
stored one or more program participants skills for job duties data and related stored configuration data;
stored one or more learning resources for skills data and related stored configuration data;
stored one or more job titles assignment rules data and related stored configuration data;
stored one or more job duties assignment rules data and related stored configuration data;
stored one or more employee transfers data and related stored configuration data;
stored one or more employee exchanges data and related stored configuration data;
stored one or more transfer requests data and related stored configuration data;
stored one or more exchange requests data and related stored configuration data;
stored one or more transfer workflows data and related stored configuration data;
stored one or more exchange workflows data and related stored configuration data;
stored one or more transfer opportunities data and related stored configuration data;
stored one or more exchange opportunities data and related stored configuration data;
stored one or more transfer compensation data and related stored configuration data;
stored one or more exchange compensation data and related stored configuration data;
stored one or more skills definition data and related stored configuration data;
stored one or more employee skills data and related stored configuration data;
stored one or more program eligibility criteria data and related stored configuration data;
stored one or more transfer strategies data and related stored configuration data;
stored one or more exchange strategies data and related stored configuration data;
one or more computer-executable instructions configured to receive one or more requests for one or more transfers and/or one or more exchanges from one or more employees who need to provide one or more in-person cares to one or more elder parents based on available one or more opportunities for one or more exchanges and/or one or more transfers.

18. The method of claim 17, further comprising steps of:
determining eligibility for the one or more transfers and/or the one or more exchanges by using one or more eligibility criteria;
determining one or more transfers assignment strategies and/or one or more exchange assignment strategies when the one or more employees are eligible for the one or more transfers and/or the one or more exchanges;
determining one or more required skills after the one or more transfers and/or the one or more exchanges;
generating one or more assignments to the one or more transfers and/or the one or more exchanges, and optionally one or more skill development proposals;
sending the generated one or more assignments to the one or more transfers and/or one or more the exchanges, and/or one or more skill development proposals, for approval to one or more workflows;
generating one or more assignments data and/or one or more skill development plans when the approval is received;
monitoring progress of the generated one or more skill development plans execution based on the received data related to the progress optionally based on stored one or more rules, with or without human input;
optionally making one or more adjustments to the one or more assignments data and/or one or more skill development plans based on one or more stored rules optionally, with or without human input;
activating one or more assignments data according to the one or more assignments and/or the one or more skill development plans optionally based on one or more stored rules, with or without human input.

19. The method of claim 17, further comprising steps of:
determining existing one or more skills of the one or more employees who are requesting the one or more transfers and/or the one or more exchanges to provide one or more in-person cares to one or more elder parents;
determining each of:
(a) available one or more remote work opportunities within one or more program participants associated with the one or more employees and required one or more skills,
(b) available one or more in-person work opportunities within one or more program participants associated with the one or more employees and required one or more skills which can enable the one or more employees provide one or more in-person cares to their one or more elder parents,
(c) available one or more remote work opportunities in other one or more program participants which are not associated with the one or more employees and required one or more skills which can enable the one or more employees provide one or more in-person cares to their one or more elder parents;
determining skill difference between the determined existing one or more skills of the one or more employees and following (d) (e) (f) being within one or more thresholds:
(d) the required one or more skills for the available one or more remote work opportunities within one or more program participants associated with the one or more employees,
(e) the required one or more skills for the available one or more in-person work opportunities within one or more program participants associated with the one or more employees,
(f) the required one or more skills for the available one or more remote work opportunities in other one or more program participants which are not associated with the one or more employees;
after approval workflow assigning to the one or more employees at least one of when the determined skill difference is within limit:
(g) the determined one or more of internal remote work opportunities,
(h) the determined one or more in-person work opportunities within the one or more program participants associated with the one or more employees,
(i) the determined one or more remote work opportunities in the one or more program participants which are not associated with the one or more employees.

20. A non-transitory computer-readable medium with an executable-program stored thereon, wherein the executable-program is configured to instruct a computer/processor to perform the method of any of claim 17 to claim 19.
